(19) United States
(12) Patent Application Publication
(10) Pub. No.: US 2003/0105108 A1
(43) Pub. Date: Jun. 5, 2003

(54) Method of treating peripheral vascular diseases, peripheral neuropathies, and autonomic neuropathies

(76) Inventor: Ralph E. Wood, Moundsville, WA (US)

Correspondence Address: PFIZER INC., PATENT DEPARTMENT, MS8260-1611, EASTERN POINT ROAD, GROTON, CT, 06340, US

(21) Appl. No.: 10/311,907

(22) PCT Filed: Jun. 29, 2001
(86) PCT No.: PCT/US01/41202

(51) Int. Cl. A61K 31/519

(52) U.S. Cl. 514/252.16

(57) ABSTRACT

A method of treating a patient suffering from peripheral vascular disease, peripheral neuropathies, or autonomic neuropathies by administering a cGMP PDE5 inhibitor such as sildenafil. The method is particularly applicable to patients suffering from diabetic foot ulcers, Raynaud's Phenomenon, CREST Syndrome, erythromatosis, rheumatoid diseases, diabetic retinopathies and onychiomycosis. According to the present invention, a cGMP PDE5 inhibitor may be administered as a prophylactic to patients predisposed to develop a peripheral vascular disease, peripheral neuropathy or autonomic neuropathy.

### CROSS REFERENCE TO RELATED APPLICATIONS

This application claims the benefit of U.S. Provisional Application No. 60/215,065, filed Jun. 30, 2000, and U.S. Provisional Application No. 60/219,029, filed Jul. 18, 2000, both entitled “Method of Treating Diabetic Ulcers,” the disclosures of both of which are hereby incorporated herein in their entireties.

### FIELD OF THE INVENTION

This invention relates to the use of cyclic guanosine 3′, 5′-monophosphate type five (cGMP PDE5) inhibitors, including the compound sildenafil, for the treatment of disease related to peripheral vascular diseases, peripheral neuropathies, autonomic neuropathies, particularly the diseases which are related to diabetes.

### BACKGROUND OF THE INVENTION

Diseases, which are related to peripheral vascular disease and autonomic neuropathies are widely varied yet consistent in their chronic pathological condition and difficulty in treatment. A large number of these diseases are related to the disease diabetes mellitus. Others, although not known to be related to diabetes are similar in their physiological effects on the peripheral vascular system. Such diseases include Raynaud's Phenomenon, including CREST syndrome, autoimmune diseases, such as erythromatosis, rheumatoid diseases, and diabetic retinopathies.

Diabetes mellitus is a serious and widespread chronic disease. Studies predict that the 1996 global diabetes prevalence of 120 million should more than double to 250 million by the year 2025, primarily due to increasing age, obesity, sedentary lifestyles, and changing dietary patterns. Many serious and costly complications affect individuals suffering from diabetes mellitus, including heart disease, kidney failure, and blindness. Nevertheless, foot complications by far take the greatest toll. It is believed that 40-70% of all lower extremity amputations are related to diabetes mellitus. Additionally, approximately 85% of all diabetes-related lower extremity amputations are preceded by a foot ulcer.

Patients with diabetes mellitus are at increased risk of developing one or more foot ulcers as a result of established long-term complications of the disease, which include impaired nerve function (neuropathy) and/or ischemia.

Local tissue ischemia is a key contributing factor to diabetic foot ulceration. In addition to large vessel disease, patients with diabetes suffer further threat to their skin perfusion in at least two additional ways. First, by involvement of the non-conduit arteries, which are detrimentally affected by the process of atherosclerosis. Second, and perhaps more importantly, by impairment of the microcirculatory control mechanisms (small vessel disease). Normally, when a body part suffers some form of trauma, the body part will, as part of the body's healing mechanism, experience an increased blood flow. When small vessel disease and ischemia are present, as in the case of many diabetics, this natural increased blood flow response is significantly reduced. This fact, together with the tendency of diabetics to form blood clots (thrombosis) in the microcirculatory system during low levels of blood flow, is believed to be an important factor in ulcer pathogenesis.

Neuropathy is yet another major complication of diabetes mellitus. No well-established treatments exist for either its symptomatic treatment or for prevention of progressive decline in nerve function. Estimates, of the prevalence of neuropathy in diabetes vary widely, from a low of 5% to a high of 80%, largely due to the numerous definitions and clinical descriptions of neuropathy. Nevertheless, the additive effects of neuropathy in the suffering diabetic patient are well known and documented

The effect of the neuropathy is complex. The loss of sensory information from the foot is related to abnormal and prolonged pressure on the areas of the foot (sensory neuropathy). Motor neuropathy leads to deformity, further increasing pressure loading on the foot. In autonomic neuropathy, loss of innervation of the sweat glands results in dry skin which cracks creating an environment amenable to infection. Autonomic dysfunction contributes further by altering the distribution of micro-circulatory blood flow, directing the blood flow through shunts and away from the nutritive skin capillaries. These factors as a whole, in conjunction with foot trauma, result in skin breakdown and ulcers.

Scientists have not yet determined the mechanism that leads to nerve damage in diabetes, but it is believed to be multifactorial. These factors include genetic predisposition, metabolic and vascular abnormalities, and lack of perturbation of growth factors. The response of the peripheral nervous system to the metabolic effects of diabetes does not appear to differ between type 1 and type 2 diabetes, which suggests a likelihood of similar clinical response to therapies in the two primary forms of the disease. There seem to be a number of susceptibility factors, as yet unknown, for the development of neuropathy, which operate in the presence of hyperglycemia (high blood sugar).

Scientists have found that nerve ischemia is involved in the pathogenesis of nerve conduction. In experimental diabetic neuropathy, practitioners in the field have theorized that a decrease in nitric oxide (NO) levels may be responsible for the decrease in nerve blood flow. NO is a short-lived radical with a broad spectrum of metabolic functions, including mediation of vascular tone. The effects of NO are mediated by cyclic guanosine monophosphate (cGMP). Various therapeutic interventions, all of which increase levels of NO, have been shown to increase nerve blood flow and nerve conduction in experimental diabetic neuropathy which results in reduced levels of NO.

There are known cGMP PDE5 inhibitors, such as sildenafil citrate, which are competitive, potent, and selective inhibitors of cGMP-specific phosphodiesterase (PDE5), a compound known to be responsible for the breakdown of cGMP. Such inhibitor compounds, therefore, increase intracellular concentrations of nitric-oxide derived cGMP, thereby enhancing the effect of NO, which is responsible for the efficacy of sildenafil in the treatment of male erectile dysfunction.

While the beneficial effects of sildenafil in the treatment of erectile dysfunction have been well documented and publicized in recent years, the effectiveness of such a compound in the treatment of diabetic foot ulcers was entirely unexpected. Recent publications by Reuters™ (Reuters Health Information, Jun. 18, 2000) of the controlled study of the commercial (sildenafil) product VIAGRA™ (Pfizer) in diabetic men by Dr. Stanley Korenman, of the University of California at Los Angeles indicates an interest in the use of sildenafil for the treatment of erectile dysfunction in patients with diabetes. However, no interest in or notice of the effectiveness of cGMP PDE5 inhibitors in the treatment of diabetic foot ulcers was reported.

### SUMMARY OF THE INVENTION

Surprisingly, the inventor discovered that in treating male diabetic patients for erectile dysfunction, those that also suffered from chronic, unhealed foot ulcers achieved unexpected, rapid and complete healing of their foot ulcers. Repeated administration of the inhibiting compound to additional diabetic patients, some of whom had suffered with unhealed foot ulcers for as long as one year, achieved the same surprising results. Similar surprising results have been observed in the treatment of other disease conditions which are related to peripheral vascular disease

Clearly, the use of such inhibitor compounds represents a dramatically effective treatment of patients suffering from diabetic foot ulcers. It is known that NO is released from vascular endothelium and modulates local blood flow by relaxing vascular smooth muscle. This system is disrupted in diabetes and the increased intracellular concentrations of nitric-oxide derived cGMP, seen for example with sildenafil, therefore are believed to reverse the microvascular pathology of patients with diabetic foot ulceration leading to improved healing rates. While the present invention is not limited by this theory of physiological mechanism of the invention, the inventor believes that it is such a mechanism that the inventor's administration of sildenafil can enhance the blood supply to the ulcerated limb and thus enhance the rate of healing in diabetic foot ulcers.

It is therefore an object of the present invention to provide a method of treating a patient with diabetic ulcers, which includes treating the patient with an effective amount of a cGMP PDE5 inhibitor, or a pharmaceutical composition thereof.

It is another object of the present invention to provide a prophylactic to those patients which are predisposed to diabetic ulcers and thus save many diabetics from suffering the deleterious effects and possibility of limb amputations which commonly result from diabetic foot ulcers.

Additionally, the cGMP PDE5 inhibitor, or a pharmaceutical composition thereof, also may be used in combination with other therapeutic agents or treatments that are now or may later be useful in the treatment of the above-mentioned disease states.

The present invention also provides for the use of a cGMP PDE5 inhibitor for the manufacture of a composition for the treatment of diabetic ulcers.

It is also within the concept of this invention to treat peripheral vascular diseases such as Raynaud's Phenomenon, including CREST syndrome, autoimmune diseases such as systemic lupus erythematosis, rheumatoid diseases and diabetic retinopathies.

The present invention would also be beneficial in peripheral and autonomic neuropathies or any other disease entity that results from small vessel disease and directly large vessel disease.

Another object of this invention is the treatment of onychiomycosis (fungal) infection of the nailbed).

A number of potent and selective cGMP PDE5 inhibitors are now known and their activity can be determined readily by in-vitro screening against cGMP PDE enzymes from a number of sources, in accordance with published procedures. Thus, for example, a number of pyrazolopyrimidinone compounds are described as cGMP PDE5 inhibitors in EPO 0463756, EPO 0526004, WO 93/12095, WO 94/05661, WO 94/00453, WO 95/19978 and WO 98/49166, the complete disclosures of which are fully incorporated herein by reference.

Some cGMP-PDE5 inhibitors which can be used in the present invention include, for example, 5-(2-ethoxy-5-morpholinoacetylphenyl)-1-methyl-3-n-propyl-1-6-dihydro-7H-20 pyrazolo[4,3-d]pyrimidin-7-one; 5-(5-morpholinoacetyl-2-n-propoxyphenyl)-1-methyl-3-n-propyl-1-,6-dihydro-7H pyrazolo[4,3-d]pyrimidin-7-one; 5-[2-ethoxy-5-(4-methyl-1-piperazin-1-yl-sulphonyl)-phenyl]-1,6-dihydro-1-methyl-3 propylpyrazolo[4,3-d]pyrimidin-7-one; 5-[2-allyloxy-5-(4-methyl-1-piperazinlysulphonyl)-phenyl]-1-methyl-3-n-propyl-1,6 dihydro-7H-pyrazolo[4,3-d]pyrimidin-7-one; 5-(2-ethoxy-5-[4-(2-propyl)-1-piperazinyl-sulphonyl]phenyl}-1-methyl-3-n-propyl-1,6-dihydro-7H-pyrazolo[4,3-d)pyrimidin-7-one; 5-(2-ethoxy-5[4-(2-hydroxyethyl-1-piperazinyl-sulphonyl]phenyl)-1-methyl-3-n propyl-1,6-dihydro-7H-pyrazolo [4,3-d]pyrimidin-7-one; 5-(5-[4-(2-hydroxyethyl)-1-piperazinylsulphonyl]2-n-propoxyphenyl)-1-methyl-3-n propyl-1-,6-dihydro-7H-pyrazolo[4,3-d]pyrimidin-7-one; 5-[2-ethoxy-5-(4-methyl-1-piperazinylcarbonyl)-phenyl]-1-methyl-3-n-propyl-1,6 dihydro-7H-pyrazolo[4,3-d]pyrimidin-7-one; 5-[2-ethoxy-5-(1-methyl-2-imidazolyl)phenyl]-1-methyl-3-n-propyl-1,6-dihydro-7H pyrzolo[4,3-d]pyrimidin-7-one, 3-ethyl-5-[5-[4-ethylpiperazin-1-yl)sulphonyl]-2-(2-methoxyethoxy)pyrid-3-yl]-2-(2pyridylmethyl)-6,7-dihydro-2H-pyrazolo-[4,3-d]pyrimidin-7-one, and 3-ethyl-5-[5-(4-ethylpiperazin-1-ylsulphonyl)-2-n-propoxyphenyl]-2-(pyridin-2yl)methyl-2,6-dihydro-7H-pyrimidin-7-one.

The inventor has determined that the preferred compound, 5-[2-ethoxy-5-(4-methylpiperazin1-ylsulphonyl)-phenyl]-1,6-dihydro-1-methyl-3-propylpyrazolo[4,3-d]pyrimidine-7-one (sildenafil), and pharmaceutically acceptable salts thereof; including the citrate salt, has been very effective in the treatment of foot ulcers related to diabetes.

In regards to other uses of cycli guanosine 3′, 5′-monophosphate type five (cGMP PDE5) inhibitors, including the compound sildenafil, for which we have shown is effective in the treatment of diabetic foot ulcers, it is also within the concept of the present invention to treat other peripheral vascular diseases such as Raynaud's Phenomenon, including CREST syndrome, autoimmune diseases such as systemic lupus erythematosis, rheumatoid diseases and diabetic retinopathies. The treatment of the present invention would also be beneficial in peripheral and autonomic neuropathies or any other disease entity that results from small vessel disease and directly large vessel disease. The inventor has also discovered that onychiomycosis (fungal infection of the nailbed) particularly of the lower extremity has resolved completely without the use of antifungal medication when treated exclusively with sildenafil. The patient in this case was requesting treatment for erectile dysfunction and again the inventor discovered this unexpected beneficial result. The inventor has observed such results in varied diseases which have the common element of peripheral vascular disease or peripheral neuropathy. The beneficial effect of the method is believed to be due to increase vascular flow of the small vessels which aided the body in healing itself.

### EXAMPLES

Patient #1 is an insulin dependent diabetic who had been suffering from erectile dysfunction and who subsequently had a diabetic foot ulcer. During the treatment of the erectile dysfunction it was noted that the foot ulcer was healing. This foot ulcer began approximately two years prior and the patient had been through vascular studies, had seen vascular surgeons, podiatrists, and had been in wound care clinics with minimal results at best. He had also been hospitalized for approximately a month on IV antibiotics, etc. and the threat was very real that the patient was going to require a below the knee amputation. The ulcer would appear to be healing at times only to reoccur to its pretreatment size and depth. Once sildenafil treatment had began for his erectile dysfunction, it was noted that the ulcer was decreasing in the size and the patient was instructed to begin taking 50 mg of sildenafil once a day. This resulted in complete resolution of the diabetic food ulcer in one month and the patient has continued on this same treatment for the past two years without reoccurrence.

Patient #2 was suffering from chronic changes of both lower extremities secondary to peripheral vascular disease and diabetes mellitus. He was being followed for his diabetes mellitus and stated he was having trouble with erectile dysfunction and once sildenafil treatment was instituted, not only did his erectile dysfunction significantly improve but the chronic changes of both lower extremities secondary to the peripheral vascular disease also significantly improved or resolved completely.

Patient #3 suffers from severe peripheral vascular disease secondary to arteriosclerosic. Conventional treatments such as femoral popliteal bypass surgery, surgical insertion of (Greenfield filter) thrombotic preventive umbrella, administration of heparin and administration of coumadin have all failed to alleviate the condition. Sildenafil has been prescribed for erectile dysfunction and the patient is being closely followed to monitor improvements in the arteriosclerosic condition.

Patient #4 suffered from erectile dysfunction. He also suffered from onychiomycosis (fungal infection of the nailbed). He was placed on sildenafil treatment taking one 50 mg pill on an as-needed basis. On that treatment schedule his erectile dysfunction improved and surprisingly his fungal infection was cured.

The cGMP PDE5 inhibitor is preferably administered as a pharmaceutical composition. Thus, the compound can be administered in any conventional oral, parenteral, rectal, or transdermal dosage form, usually with a pharmaceutically acceptable carrier or diluent. These methods of administration are well known in the prior art and are disclosed in U.S. Pat. Nos. 5,520,534; 5,346,901; 5,719,283; 5,272,147; 5,426,107; 5,482,941; 5,591,742; 5,734,053; 6,025,494; 5,859,006, the complete disclosures of which are fully incorporated herein by reference.

Oral administration of a pharmaceutical composition may be in the form of a solution, suspension, tablet, pill, capsule, powder or the like. Tablets containing various excipients such as sodium citrate, calcium carbonate and calcium phosphate are used in conjunction with various disintegrants, such as potato or tapioca starch, and certain complex silicates, together with binding agents such as polyvinylpyrrolidone, sucrose, gelatin, and acacia. Additionally, lubricating agents such as magnesium stearate, sodium lauryl sulfate, and talc are often used for tabletting purposes. Solid compositions of a similar type are also employed as fillers in soft and hard-filled gelatin capsules; preferred materials in this connection also include lactose or milk sugar, as well as high molecular weight polyethylene glycols. When aqueous suspensions and/or elixirs are desired for oral administration, the compounds can be combined with various sweetening agents, flavoring agents, coloring agents, emulsifying agents and/or suspending agents, as well as such diluents as water, ethanol, propylene glycol, glycerin and various similar combinations thereof. It is also within the concept of the present invention to administer the effective compound in admixture with a foodstuff or drink.

For purposes of parenteral administration, solutions in oil or in aqueous propylene glycol can be employed, as well as sterile aqueous solutions of the corresponding water-soluble salts. Such aqueous solutions may be suitably buffered, if necessary, and the liquid diluent first rendered isotonic with sufficient saline or glucose. These aqueous solutions are especially suitable for intravenous, intramuscular, subcutaneous and intraperitoneal injection purposes. In this connection, the sterile aqueous media employed are all readily obtainable by standard techniques, which are well-known to those skilled in the art.

For purposes of transdermal (e.g., topical) administration, dilute sterile, aqueous or partially aqueous solutions (usually in about 0.1% to 5% concentration), otherwise similar to the above parenteral solutions, are prepared. Transdermal administration of compounds for therapeutic purposes is increasingly becoming a common practice, as in the case of nicotine patches or motion sickness preventatives. More recently, the transdermal application of relatively large molecules, such as those contained in compositions of antigens and adjuvants has been shown to be effective as described in U.S. Pat. No. 5,910,306 and U.S. Pat. No. 5,980,898, the complete disclosures of which are fully incorporated herein by reference. Transdermal application can be accomplished by direct application to the skin, in admixture with a carrier, such as for example a salve or cream, or as covered by or applied to a patch, which is placed on the skin of the patient.

Methods of preparing various pharmaceutical compositions with a certain amount of active ingredient are well known to those skilled in the art, or may be determined by reference to literature precedents, which are available to those skilled in the art.

The exact dosages of cGMP PDE5 inhibitor administered will differ depending upon the specific compound prescribed, on the subject being treated, on the severity of the condition, on the manner of administration, and on the judgment of the prescribing physician. Thus, because of patient-to-patient variability, dosages are a guideline only and the physician may adjust doses of the compounds to achieve the level of effective treatment that the physician considers appropriate for the patient. In considering the degree of treatment desired, the physician must balance a variety of factors such as the age of the patient and the presence of other diseases or conditions (e.g. cardiovascular disease). In general, the cGMP PDE5 inhibitor will be administered in a range of from 0.5 to 400 mg per day, as disclosed in disclosed in U.S. Pat. Nos. 5,520,534; 5,346,901; 5,719,283; 5,272,147; 5,426,107; 5,482,941; 5,591,742; 5,734,053; 6,025,494; 5,859,006, the complete disclosures of which are fully incorporated herein by reference. More particularly, the preferred treatment dosage is 25 to 100 mg per day, as disclosed in WO 98/49166, the complete disclosure of which is fully incorporated herein by reference. Safe and effective dosages prescribed for individuals would be well known to a practitioner, based upon the disclosures in the foregoing patents and the practitioner's personal knowledge of the particular patient's state of health.

In the case of the preferred compound, sildenafil, an effective dose is 5 to 125 mg per day, more preferably 10-110 mg per day and most preferably 25-100 mg per day, which can be administered as a tablet or capsule up to three times a day. However, the precise dosage will be as determined by the prescribing physician and will depend on the age and weight of the patient and severity of the symptoms, as described above.

The administration of an effective dosage of the compound of the present invention can also provide a prophylactic to assist in the prevention of foot ulcers in a patient suffering from the disease diabetes. The skilled practitioner can prescribe an effective dosage of the compound using the dosages disclosed above and tailored to the specific needs of the patient being treated.

What I claim is:

1. A method of treating the disease of a type selected from peripheral vascular diseases, peripheral neuropathies, and autonomic neuropathies, comprising administering an effective amount of a pharmaceutical composition comprising a cGMP PDE5 inhibitor or a derivative or salt thereof.

2. A method of treating a disease according to claim 1 wherein said disease is selected from the group consisting of Raynaud's Phenomenon, CREST syndrome, erythromatosis, rheumatoid diseases and diabetic retinopathies.

3. A method of treating onychiomycosis comprising administering an effective amount of a pharmaceutical composition comprising a cGMP PDE5 inhibitor or a derivative or salt thereof.

4. A method of treating a patient suffering from diabetic foot ulcers which comprises treating said patient with an effective amount of a pharmaceutical composition comprising a cGMP PDE5 inhibitor or a derivative or salt thereof.

5. A method of preventing the formation of foot ulcers in a patient suffering from the disease diabetes, the method comprising administering to said patient an effective amount of a pharmaceutical composition comprising a cGMP PDE5 inhibitor or a derivative or salt thereof.

6. A method of manufacturing a diabetic foot ulcer therapeutic comprising providing an effective ingredient selected from the group consisting of a cGMP PDE5 inhibitor, a derivative thereof, and a salt thereof and combining said effective ingredient with a suitable carrier for administration to a patient.

7. A method according to claim 4 or 5, wherein the cGMP PDE5 inhibitor is sildenafil.
